(19) United States
(12) Patent Application Publication
(10) Pub. No.: US 2008/0114344 A1
(43) Pub. Date: May 15, 2008

(54) Closed Loop Cryosurgical System

(76) Inventors: Jia Hua Xiao, Santa Rosa, CA (US); Ling Wang, Plymouth, MN (US)

Correspondence Address: AMS RESEARCH CORPORATION, 10700 BREN ROAD WEST, MINNETONKA, MN, 55343, US

(21) Appl. No.: 11/938,898

(22) Filed: Nov. 13, 2007

Related U.S. Application Data
(60) Provisional application No. 60/865,524, filed on Nov. 13, 2006.

(51) Int. Cl. A61B 18/02

(52) U.S. Cl. 606/20

(57) ABSTRACT

A cryosurgical system and related methods of utilizing distinct freeze and thaw fluids to selectively freeze or thaw tissue as part of a cryosurgical treatment plan. A closed loop cryosurgical system can include a control console, a cryocooler, a freeze tank, a thaw tank as well as fluid supply and fluid return lines operably interconnecting one or more cryoprobes with the freeze and thaw tank. At the direction of the control console, the cryocooler is used to cool a freeze fluid in the freeze tank while a heating element in the thaw tank is used to head a thaw fluid in the thaw tank. By using distinctly controlled reservoirs of freeze and thaw fluid, the cryosurgical system is able to quickly cycle back and forth between freeze and thaw cycles conducted as part of a cryosurgical treatment.

### PRIORITY CLAIM

The present application claims priority to U.S. Provisional Application Ser. No. 60/865,524, filed Nov. 13, 2006 and entitled “CLOSED LOOP CRYOSURGICAL SYSTEM”, which is herein incorporated by reference in its entirety.

### FIELD OF THE INVENTION

The present disclosure is directed toward cryoablation treatment of the prostate. In particular, the present disclosure is directed to a cryosurgical system utilizing distinct freeze and thaw fluids for selectively freezing or thawing prostate tissue as part of cryosurgical treatment plan.

### BACKGROUND OF THE INVENTION

Cryoablation of the prostate involves controlled freezing of portions of the prostate to selectively kill cancerous tissue as well as the connective tissue and capillaries surrounding the cancerous tissue. When exposed to freezing, the cancerous cells are destroyed while the destruction of the surrounding connective tissue and capillaries prevents and/or inhibits any additional growth of the cancerous tissue. Cryosurgical probes quickly freeze diseased body tissue, causing the tissue to die after which it will be absorbed by the body, expelled by the body, sloughed off or replaced by scar tissue. In addition to treatment of the prostate, cryoablation has been used successfully in a variety of gynecological applications as well as for treatment of a number of other diseases and conditions including breast cancer, liver cancer, renal cancer glaucoma and other eye diseases.

A variety of cryosurgical instruments variously referred to as cryoprobes, cryosurgical probes, cryosurgical ablation devices, cryostats and cryocoolers have been used as patient interfaces during cryosurgery. These devices typically use the principle of Joule-Thomson expansion to generate cooling. The devices take advantage of the fact that most fluids, when rapidly expanded, become extremely cold. In these devices, a high pressure gas mixture is expanded through a nozzle inside a small cylindrical shaft or sheath typically made of steel. The Joule-Thomson expansion cools the steel sheath to a cold temperature very rapidly. The cryosurgical probes then form ice balls which freeze diseased tissue. A properly performed cryosurgical procedure allows cryoablation of the diseased tissue without undue destruction of surrounding healthy tissue.

### SUMMARY OF THE INVENTION

The present disclosure is directed to a cryosurgical system and related methods of utilizing distinct freeze and thaw fluids to selectively freeze or thaw tissue as part of a cryosurgical treatment plan. A closed loop cryosurgical system can comprise a control console, a cryocooler, a freeze tank, a thaw tank as well as fluid supply and fluid return lines operably interconnecting one or more cryoprobes with the freeze and thaw tank. At the direction of the control console, the cryocooler is used to cool a freeze fluid in the freeze tank while a heating element in the thaw tank is used to heat a thaw fluid in the thaw tank. By using distinctly controlled reservoirs of freeze and thaw fluid, the cryosurgical system is able to quickly cycle back and forth between freeze and thaw cycles conducted as part of a cryosurgical treatment.

In one aspect, the present disclosure is directed to a cryosurgical system having distinct reservoirs of freeze and thaw fluids that are monitored and adjusted by a control console. The freeze and thaw fluids can be selectively routed through one or more cryoprobes to conduct ablation and thaw cycles during a cryosurgical treatment, i.e., each cryoprobe is controlled independently from the others.

In another aspect, the present disclosure is directed to a method for conducting a cryosurgical treatment wherein distinct freeze and thaw fluids are monitored and adjusted such that the freeze and thaw fluids can be directed through one or more cryoprobes to conduct freeze and thaw portions of the cryosurgical treatment.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE FIGURES

These as well as other objects and advantages of this invention, will be more completely understood and appreciated by referring to the following more detailed description of the presently preferred exemplary embodiments of the invention in conjunction with the accompanying drawings of which:

FIG. 1 is a schematic illustration of an embodiment of a closed loop cryosurgical system according to the present disclosure.

### DETAILED DESCRIPTION

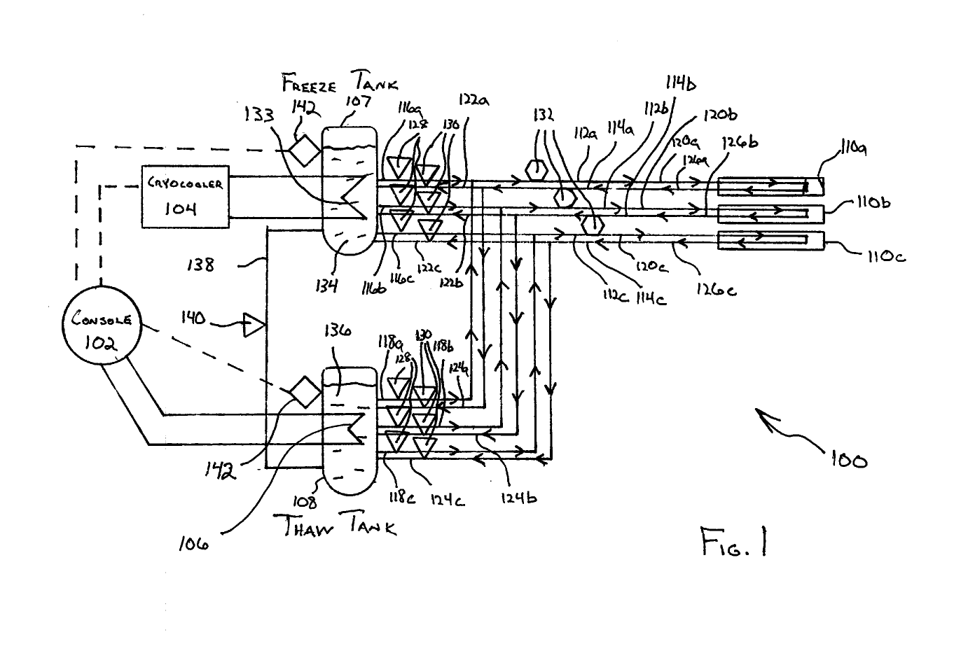

Referring to FIG. 1, there can be seen an embodiment of a closed loop cryosurgical system 100 according to the present disclosure. Cryosurgical system 100 generally includes a control console 102 operably interconnected with a cryocooler 104 and a heating element 106. An exemplary control console 102 that may be used with an embodiment of the present invention is used as part of the Her Option® Office Cryoablation Therapy available from American Medical Systems of Minnetonka, Minn. Console 102 can include controls that allow for monitoring, activation, deactivation, and modification of various system parameters, such as the flow rates, pressures, and temperatures of the refrigerants. In addition, console 102 can include a display that allows the operator to quickly monitor, and in some embodiments adjust, the performance of cryosurgical system 100.

In one representative embodiment, cryocooler 104 can comprise a dual-stage compressor system with a primary compressor providing a primary pressurized, mixed gas refrigerant and a secondary compressor providing a secondary pressurized, mixed gas refrigerant. Through the use of a dual-stage compressor system, cryocooler 104 can achieve cooler temperatures than a single stage compressor system. In addition, the use of gas mixtures for the primary pressurized, mixed gas refrigerant and secondary pressurized, mixed gas refrigerant are known in the art that provide a dramatic increase in cooling performance over a single gas.

Referring again to FIG. 1, cryosurgical system further comprises a freeze tank 107, a thaw tank 108 and a plurality of cryoprobes 110a, 110b and 110c. Cryoprobes 110a, 110b and 110c are fluidly interconnected to the freeze tank 107 and thaw tank 108 with supply fluid lines 112a, 112b and 112c and return fluid lines 114a, 114b, 114c. The supply fluid lines 112a, 112b, 112c and return fluid lines 114a, 114b, 114c are generally flexible to allow for easy manipulation and placement of cryoprobes 110a, 110b and 110c. In addition, supply fluid lines 112a, 112b and 112c and return fluid lines 114a, 114b, 114c can be insulated so as to limit heat transfer with the ambient environment and prevent medical professional from being exposed to potentially uncomfortable or unsafe temperatures.

Supply fluid lines 112a, 112b and 112c each comprise an ablation supply portion 116a, 116b, 116c, a thaw supply portion 118a, 118b, 118c and a cryoprobe supply portion 120a, 120b, 120c while return fluid lines 114a, 114b, 114c comprise an ablation return portion 122a, 122b, 122c, a thaw return portion 124a, 124b, 124c and a cryoprobe return portion 126a, 126b, 126c. Each of the ablation supply portions 116a, 116b, 116c and thaw supply portions 118a, 118b, 118c include a supply valve 128 while each of the ablation return portions 122a, 122b, 122c and thaw return portions 124a, 124b, 124c include a return valve 130. Supply valve 128 and return valve 130 can comprise suitable manual valve or remotely actuated, automated valves. In one representative embodiment, the supply and return valves can comprise solenoid valves that are operably interconnected with the control console 102 such that actuation of each valve is independently controlled by the control console 102. In addition, each of the cryoprobe supply portions 120a, 120b, 120c includes a cryoprobe supply pump 132.

Freeze tank 107 and thaw tank 108 generally comprise sealed and insulated tanks fabricated of materials suitable for use with extreme cold temperatures as well as elevated temperatures. Freeze tank 107 includes a cooling heat exchanger 133 that is operably interconnected to the cryocooler 104. Freeze tank 107 includes a reservoir of an ablation fluid 134 and the thaw tank includes a reservoir of a thaw fluid 136. Ablation fluid 134 and thaw fluid 136 can comprise any suitable heat exchange fluid wherein ablation fluid 134 has desirable freeze properties and thaw fluid 136 has desirable boiling properties. In some instances, ablation fluid 134 and thaw fluid 136 can comprise the same fluid as long as the freezing and boiling properties are suitable for the desired temperature operation range of the cryosurgical system 100. Representative heat exchange fluids can comprise perfluorocarbon (PFC) fluids as well as Asahikling 225 (AK-225) commercially available from the Asahi Glass Company. When ablation fluid 134 and thaw fluid 136 comprise the same fluid, an exchange line 138 can operably, fluidly connect the freeze tank 107 and thaw tank 108. Exchange line 138 can comprise an exchange valve 140 for isolating the freeze tank 107 and thaw tank 108 during a cryosurgical treatment. Both freeze tank 107 and thaw tank 108 can also comprise temperature control sensors 142 operably connected to the control console 102.

Cryoprobes 110a, 110b and 110c can comprise any of a variety of different cryoprobes selected based upon the cryosurgical treatment to be performed. In some representative embodiments, cryoprobes 110a, 110b, 110c can be rigid or flexible, straight or curved, long or short and the like. Furthermore, cryoprobes 110a, 110b, 110c can be selected for specific cryosurgical treatment applications including, for example, urethra treatment, prostate treatment, bladder treatment and ureter/kidney treatment. Although not presently illustrated, it will be understood that cryosurgical system 100 can further comprise additional components for assisting with the maintenance and positioning of cryoprobes 110a, 110b and 110c. In some embodiments, cryoprobes 110a, 110b, 110c can include temperature sensors that are in operable communication with the control console 102 for monitoring and controlling cryoprobe temperature during treatment. For instance, the supply fluid lines 112a, 112b, 112c, return fluid lines 114a, 114b, 114c and cryoprobes 110a, 110b, 110c connect to the freeze tank 107 and thaw tank 108 by way of an articulating arm, which may be manually or automatically used to position the cryostats 110a, 110b, 110c. In some embodiments, the articulating arm may incorporate the supply fluid lines 112a, 112b, 112c and return fluid lines 114a, 114b, 114c within the articulating arm. In addition, the cryosurgical system can further include a positioning grid to properly align and position the cryoprobes 110a, 110b and 110c for patient insertion.

In use, a medical professional determines the desired operating conditions of the cryosurgical system 100 and inputs them into the control console 102. In general, a cryosurgical treatment will consist of at least one cryoablation cycle. Frequently, the cryosurgical treatment will include several distinct cryoablation cycles with a thaw cycle occurring between each cryoablation cycle, wherein one or more cryoprobes are operated independently in a simultaneous or sequential manner as appropriate to the application.

During a first cryoablation cycle, the control console activates the cryocooler 104 such that the freeze tank 107 can be cooled. In the case of cryocooler 104 comprising a dual-stage compressor system, a primary mixed gas refrigerant is used to cool a secondary mixed gas refrigerant, which is subsequently pumped through the cooling heat exchanger 133. As the secondary mixed gas refrigerant passes through the cooling heat exchanger 133, heat energy is transferred from the ablation fluid 134 into the secondary mixed gas refrigerant such that over a relatively short period of time, the temperature of the ablation fluid 134 approaches the incoming temperature of the secondary mixed gas refrigerant. Once the ablation fluid 134 reaches the desired ablation temperature as measured by the temperature control sensor 142, ablation fluid 134 is ready to supply one or more of the cryoprobes 110a, 110b, 110c.

For purposes of describing use of the cryosurgical system 100, the use of cryoprobe 110a will be described though it is to be understood that the same principles of operation apply to the use of cryoprobes 110b and 110c as well. When the medical professional is ready to utilize cryoprobe 110a during an ablation cycle, the supply valves 128 within the ablation supply portion 112a and ablation return portion 122a are opened while the supply valves 128 within the thaw supply portion 118a and the thaw return portions 124a are maintained in a closed position. Next, the cryoprobe supply pump 132 within the cryoprobe supply portion 120a is actuated such that ablation fluid 134 is pumped through the supply fluid line 112a and into the cryoprobe 110a. Within the cryoprobe 110a, the ablation fluid 134 flows into a Joule-Thompson expansion element, such as a valve, orifice, or other type of flow constriction, located near the tip of the cryoprobe 110a, where the ablation fluid 134 is expanded isenthalpically to a lower temperature. A typical Joule-Thompson expansion element is a capillary tube. The ablation fluid 134 then cools a heat transfer element mounted in the wall of the cryoprobe 110a so at to form ice ball at the tip of the cryoprobe 110a that is used to freeze diseased tissue. The ablation fluid 134 then returns to the freeze tank 107 through the cryoprobe return portion 126 and the ablation return portion 122a. The ablation fluid 134 then returns to the freeze tank 107 where its temperature is again lowered by exposure to the cooling heat exchanger 133.

Following the ablation cycle, the control console activates the heating element 106 to begin warming the thaw fluid 136 within the thaw tank 108. Heating element 106 can comprise a resistive heating element wherein current supplied to the heating element 106 is converted to heat energy used to heat the thaw fluid 136. Using temperature control sensor 142, the control console 102 monitors and controls the operation of the heating element 106 such that the thaw fluid 136 reaches and is maintained at a desired temperature.

When the medical professional is ready to utilize cryoprobe 110a during the thaw cycle, the supply valves 128 within the thaw supply portion 118a and thaw return portion 124a are opened while the supply valves 128 within the ablation supply portion 116a and the ablation return portion 124a are closed. Once again, the cryoprobe supply pump 132 within the cryoprobe supply portion 120a is actuated such that the thaw fluid 136 is pumped through the cryoprobe supply portion 120a and into the cryoprobe 110a. Within the cryoprobe 110a, the thaw fluid 136 warms the heat transfer element mounted in the wall of the cryoprobe 110a so at to melt the ice ball at the tip of the cryoprobe 110a and eventually thaw the previously frozen tissue. This allows the tip of the cryoprobe 110a to be removed from the tissue without causing further damage to healthy tissue. The thaw fluid 136 then returns to the thaw tank 108 through the cryoprobe return portion 126a and the thaw return portion 124a. The thaw fluid 136 returns to that tank 108 where its temperature is again increased by exposure to the heating element 106. Upon completion of a thaw cycle, cryoprobe 110a can be removed from the treatment area if treatment is complete or alternatively, the medical professional can commence a new freeze cycle.

As will be understood by one of skill in the art, cryosurgical system 100 can comprise a variety of physical configurations wherein various components can be grouped together to form combined units or distinct portions of the overall system. For example, cryosurgical system 100 can take the form of a cabinetized or skid-mounted assembly wherein the major components including the control console 102, cryocooler 104, freeze tank 107, and thaw tank 108 are assembled and packaged as a single, unitized assembly. Alternatively, the cryosurgical system 100 can comprise a plurality of distinct assemblies such as, for example, an assembly comprising the control console 102 and cryocooler 104 and a second assembly comprising the freeze tank 107 and thaw tank 108.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

What is claimed is:

1. A closed loop cryosurgical system comprising:
a control console operably connected to a cryocooler and a heating element;
a freeze tank including an ablation fluid and a cooling heat exchanger, wherein the cooling heat exchanger is operably connected to the cryocooler and configured to cool the ablation fluid;
a thaw tank including a thaw fluid, wherein the heating element is contained within the thaw tank, said heating element being configured to heat the thaw fluid; and
a plurality of cryoprobes, each cyroprobe individually fluidly interconnected to a supply fluid line and a return fluid line, wherein the supply fluid lines and the return fluid lines are fluidly connected to the freeze tank and the thaw tank, and wherein each of said plurality of cryoprobes is independently operable.

2. The system of claim 1, wherein the supply fluid lines each comprise an ablation supply portion, a thaw supply portion, and a cryoprobe supply portion and the return fluid lines each comprise an ablation return portion, a thaw return portion, and a cryoprobe return portion.

3. The system of claim 2, wherein each ablation supply portion and thaw supply portion include a supply valve and wherein each ablation return portion and thaw return portion include a return valve configured to allow or prevent fluid flow through each respective portion, and wherein the supply valves and the return valves are selectively controllable by the control console.

4. The system of claim 2, wherein each of said plurality of cryoprobes operates independently and in a simultaneous or sequential manner.

5. The system of claim 1, wherein the supply fluid lines and return fluid lines are insulated.

6. The system of claim 1, wherein the ablation fluid and the thaw fluid comprise different heat exchange fluids.

7. The system of claim 1, wherein the ablation fluid and the thaw fluid are a common heat exchange fluid.

8. The system of claim 7, further comprising an exchange line fluidly interconnecting the freeze tank and the thaw tank.

9. The system of claim 8, wherein the exchange line includes an exchange valve configured to selectively isolate the freeze tank and the thaw tank during a cryosurgical treatment.

10. The system of claim 1, wherein the freeze tank and thaw tank each include a temperature control sensor operably connected to the control console for controlling operation of the cyrocooler and the heating element.

11. The system of claim 1, wherein the cryocooler comprises a dual-stage compressor system having a primary compressor for pressurizing a primary refrigerant and a secondary compressor for pressurizing a secondary refrigerant.

12. The system of claim 1, wherein the heating element comprises a resistive heating element.

13. A method of utilizing an ablation fluid and a thaw fluid to perform a cryosurgical treatment comprising:
activating a cryocooler to cool an ablation fluid in a freeze tank;
activating a heating element to warm a thaw fluid in a thaw tank;
supplying the cooled ablation fluid to one or more cryoprobes through supply fluid lines;
freezing selected tissue with the cryoprobes;
returning the ablation fluid to the freeze tank through return fluid lines to be recooled;
supplying the heated thaw fluid to the cryoprobes through the supply fluid lines;
thawing selected frozen tissue with the cryoprobes; and
returning the thaw fluid to the thaw tank through the return fluid lines to be reheated.

14. The method of claim 13, further comprising:
supplying the recooled ablation fluid to the cryoprobes;
refreezing the selected tissue with the cryoprobes;
returning the ablation fluid to the freeze tank to be recooled again;
supplying the reheated thaw fluid to the cryoprobes;
rethawing the frozen tissue with the cryoprobes; and
returning the thaw fluid to the thaw tank to be reheated again.

15. The method of claim 13, wherein the cryocooler cools the ablation fluid with a secondary refrigerant that has been precooled by a primary refrigerant.

16. The method of claim 13, further comprising:
opening an ablation supply valve and an ablation return valve and closing a thaw supply valve and a thaw return valve prior to supplying the ablation fluid to the cryoprobes.

17. The method of claim 16, further comprising:
opening the thaw supply valve and the thaw return valve and closing the ablation supply valve and the ablation return valve prior to supplying the thaw fluid to the cryoprobes.

18. The method of claim 13, wherein supplying the ablation fluid to the one or more cryoprobes comprises individually pumping the ablation fluid to the one or more cryoprobes.

19. The method of claim 18, wherein supplying the thaw fluid to the one or more cryoprobes comprises individually pumping the thaw fluid to the one or more cryoprobes.

20. The method of claim 13, further comprising monitoring the temperatures of the ablation fluid and the thaw fluid.
